(19) United States
(12) Patent Application Publication
(10) Pub. No.: US 2006/0199876 A1
(43) Pub. Date: Sep. 7, 2006

(54) Bioceramic composite coatings and process for making same

(75) Inventors: Tomasz Troczynski, Vancouver (CA); Quanzu Yang, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: The University of British Columbia, Vancouver (CA)

(21) Appl. No.: 11/071,264

(22) Filed: Mar. 4, 2005

(51) Int. Cl. A61K 6/083

(52) U.S. Cl. 523/115

(57) ABSTRACT

The present invention discloses novel polymer-ceramic matrix composites and processes for making same. The composites can be used in biomedical applications, in particular, coatings of implants and other medical devices, where both the ceramic phase and the polymer phase are bio-compatible. The composites combine a reinforcing polymer phase with a continuous ceramic matrix to create materials with properties that are new and superior to polymer or ceramic phases alone. The composites can incorporate a bioactive agent.

### FIELD OF THE INVENTION

The present invention discloses novel polymer-ceramic matrix composites and processes for making same. The composites can be used in biomedical applications, in particular, coatings of implants and other medical devices, where both the ceramic phase and the polymer phase are bio-compatible. The composites combine a reinforcing polymer phase with a continuous ceramic matrix to create materials with properties that are new and superior to polymer or ceramic phases alone.

### BACKGROUND OF INVENTION

Bioceramics are ceramic materials used for biomedical applications. Bioceramics can be used for structural functions, e.g. for joint or tissue replacement, or can be used as coatings to improve biocompatibility of metal implants, or can function as a resorbable vehicle which provides a temporary framework that is dissolved and replaced as the body rebuilds tissue. Some bioceramics additionally feature drug-delivery capability.

Calcium phosphate (CP), in particular hydroxyapatite (HAP), are the most important inorganic constituents of biological hard tissues. In the form of carbonated HAP combined with organic component (e.g. collagen), they are present in bone, teeth, and tendons to give these organs stability, hardness, and the specific structural function. Biologically formed calcium phosphates are often nanocrystals that are precipitated under mild conditions, i.e. ambient pressure, and near room temperature. The beneficial biocompatible properties of hydroxyapatite (HAP) are well documented. HAP is rapidly integrated into the human body, e.g. it will bond to bone. Hydroxyapatite is used as a coating for implants (e.g. titanium or stainless steels). Recent studies have examined the possibility of the use of HAP in composite form, namely in materials that combine polymers with ceramic or metal/ceramic combinations. Reports of this research are available through several publications, e.g. Ritzoulis et al, “Formation of hydroxyapatite/biopolymer biomaterials. I. Microporous composites from solidified emulsions”, in Journal of Biomedical Materials Research (2004 Dec. 15), 71A(4), 675-8; Haris et al, “Nanocrystalline hydroxyapatite-polyaspartate composites”, in Bio-Medical Materials and Engineering (2004), 14(4), 573-579; Furuzono et al, “Nano-scaled hydroxyapatite/polymer composite IV. Fabrication and cell adhesion properties of a three-dimensional scaffold made of composite material with a silk fibroin substrate to develop a percutaneous device”, in Journal of Artificial Organs (2004), 7(3), 137-144.

Considerable research has been also performed on methods of producing HAP coatings on metal substrate for medical device applications, for example, WO 2004/024201, US 2002/155144, U.S. Pat. No. 6,426,114, JP 2003/342113 and US 2003/099762. Another example of inorganic biomaterial is bioglass, an oxide glass including silicon dioxide, calcium oxide and phosphorous oxide. Bioglass, including glass and glass-ceramics, is currently used as implant materials, i.e. refer to Dubok, “Bioceramics—yesterday, today, tomorrow”, in Powder Metallurgy and Metal Ceramics, Vol.39, Nos. 7-8, 2000 and Kokubo, “Surface chemistry of bioactive glass ceramics” in Journal of Non-Crystalline Solids (1990), 120(1-3), 138-51.

Chemical and morphological similarly between natural bone and the implant material tends to promote implant/bone interfacial bonding, thereby providing high interface sheer strength. The body of the patient will tend to isolate the implant if the body views the implant as foreign material, often by re-absorption of the surrounding tissue and the subsequent formation of a fibrous tissue membrane at the interface between the implant and the natural bone. Such fibrous tissue formation at the interface interferes with the development of a strong mechanical interlock between the implant and the bone material surrounding the defect site. A better interface may be achieved when the implant material either allows or even promotes bone ingrowth into the defect site, providing a superior mechanical lock with the implant or prosthesis. Various synthetic bone substitutes have been proposed, including poorly crystalline hydroxyapatite (PC-HAP), as described by Lee et al., in U.S. Pat. No. 6,331,312. Tricalcium phosphate (TCP), PC-HAP and TCP have been reported to provide implants with bioactive surfaces that promote ingrowth of natural bone when implanted into bone. In addition, it has been observed that both PC-HAP and TCP are reabsorbed by the host tissue, i.e. Seed Matsushita et al, “A new bone-inducing biodegradable porous tricalcium phosphate”, in Journal of Biomedical Materials Research, Part A (2004), 70A(3), 450-458; and Lu et al, “The biodegradation mechanism of calcium phosphate biomaterials in bone”, in Journal of Biomedical Materials Research (2002), 63(4), 408-412.

In addition to bioceramic materials, organic polymers have been used as bone defect repair materials, including poly(methyl methacrylate) (PMMA), poly(lactic acid) (PLA), and poly(glycolic acid) PGA, i.e. refer to Kaito et al, “Potentiation of the activity of bone morphogenetic protein-2 in bone regeneration by a PLA-PEG/hydroxyapatite composite”, Biomaterials (2004), Volume Date 2005, 26(1), 73-79.

PMMA, also commonly used as a bone cement, is not subject to degradation by most biological processes in the patient. However, PMMA-based compositions have been made partially resorbable by including cross-linked poly(propylene glycol fumarate) (PPF) and a particulate bioceramic, as described by Gerhart et al., in U.S. Pat. Nos. 5,085,861 and 4,843,112. However, these cements are primarily designed to be used in conjunction with the implantation of other non-resorbable prosthetic devices.

Bioceramic-polymer matrix composites are a new generation of implantation material based on calcium phosphates. They substantially expanded the possibility of restorative and substitutive osteoplastic surgery, mainly in dentistry, maxillofacial surgery, and neurosurgery. The composite of HAP and biodegradable polymer improves the mechanical strength and resistance to impact loading. In addition, HAP significantly improves the biocompatibility, bioactivity, bioresorpation of the overall composite including biopolymer. The evolution in mechanical properties due to biodegradation of the polymer can provide progressive load transfer from implant to the bone during healing, thereby eliminating stress shielding.

These types of composites are expected to be used for defect filling, augmentation of implant attachment to bone and internal fracture fixation without the use of other additional components, e.g. refer to Durucan, et al, “Biodegradable hydroxyapatite-polymer composites”, Advanced Engineering Materials (2001), 3(4), 227-231; Beletskii et al, “Biocomposite calcium-phosphate materials used in osteoplastic surgery”, in Glass and Ceramics (Translation of Steklo i Keramika) (2000), 57(9-10), 322-325.

Wang et al (Annales de Chimie (Paris, France) (2004), 29(1), 17-28) 30 reported that hydroxyapatite (HAP) and tricalcium phosphate (TCP) have been incorporated into polyhydroxybutyrate (PHB) to form new composites for tissue replacement and regeneration applications. SEM examination showed that the earliest nucleation of mineral crystals occurred on HA/PHB composites only after one day immersion in SBF.

Cooper (WO 2004/067052) discloses a method of forming a bioabsorbable implant from a composite of a bioabsorbable polymer and a bioactive ceramic filler. The surface of the implant is abraded with a biocompatible abrasive material such as a hydroxyapatite grit. A part of the outer surface of the implant is provided by the ceramic filler.

Several disclosures have been made also by W. Bonefield et al (U.S. Pat. Nos. 5,017,627, 5,728,753, 5,962,549), wherein discontinuous bio-ceramic or bio-glass particles are dispersed in bio-polymer matrix, to form bulk composites suitable for implants. However, properties of all composites wherein the polymer is the continuous phase, are controlled by the properties of the polymer phase. In particular, resorption of the polymer would lead to degradation of the whole composite.

The ideal material for medical applications would not only be biocompatible, but would also have physical properties similar to those of the tissue being replaced or repaired. Ceramics, though they include good chemical and corrosion-resistant properties, are notoriously brittle, e.g. of fracture toughness of the order of 1 MPa√m. This means that ceramics have a very low tolerance of crack-like flaws. The absence of energy-dissipating mechanisms, such as generation and movement of dislocations in ceramics, causes ceramics to fail in a catastrophic fashion. Improving the toughness of ceramics is a current research goal. One of the important approaches to accomplish this goal is via ceramic matrix composites.

Several reports describe a combination of bioceramics and biopolymer phases for increased mechanical properties of the bulk composite. For example, as reported by Komlev, et al. in “Strength enhancement of porous hydroxyapatite ceramics by polymer impregnation”, in Journal of Materials Science Letters, 22, 2003, 1215-121, disc samples of 10 mm diameter and about 4 to 6 mm thickness were uniaxially pressed at 50 MPa pressure at room temperature. The green bodies were sintered at 1200° C. for 1 h in air. The samples of porous HA ceramics were immersed in the polymer solution under a vacuum of 1.33 Pa for 10 or 30 min, and without vacuum for 30 min. The tensile strength of porous hydroyxapatite impregnated with polymer solution can be increased by a factor 2 to 6. However, this process is not suitable for making coatings on any metallic substrate for medical applications because of high temperature process for making porous ceramic body at 1200° C.

King et al (U.S. patent application Ser. No. 2004/0002770) disclose processing of polymer-bioceramic composite for orthopaedic applications. These composites are characterized by a polymer dispersed into a porous bioceramic matrix. Processes for preparing the composites by compression molding at elevated temperature are described, including compression molding to induce orientation of the polymer in multiple directions. These composites are also claimed to be useful as drug delivery vehicles to facilitate the repair of bone defects. However, there are a number of limitations to this process. For example, the molding processing of dispersed polymer at high temperature is very difficult to control because of melting polymers and the need for protecting gas environment. Additionally, the high pressure and high temperature required for the process will denature the bioactive agents if they are used as drug delivery vehicles, e.g. within the polymer matrix or ceramic matrix. Also, the high pressure processing will require high mechanical strength of porous ceramic matrix to resist the pressure without fracture. Also, this processing is not suitable for coating applications. The subject matter of the foregoing publications and patents is incorporated herein by reference.

### SUMMARY OF THE INVENTION

The present invention discloses novel polymer-ceramic matrix composites (PCMC) and processes for making same. The PCMC's are intended primarily for biomedical applications, in particular, composite coatings for medical devices. The PCMC's combine reinforcing bio-polymer phases with a bio-ceramic matrix in a unique process, to create materials that have new superior properties compared to either a polymer phase or a ceramic phase alone.

The invention is directed to a bio-polymer/bioceramic matrix composite coating comprising: (a) a porous bioceramic matrix of continuous phase; and (b) at least one biocompatible polymer of continuous or discontinuous phase. A bioactive agent can be incorporated in the composite coating.

The porous bioceramic matrix can be made by a process selected from the group consisting of sol-gel coating, thermal spray coating, electro-chemical deposition, electrophoretic deposition, biomimetic deposition and a shape and sintering process, as known in the art.

The coating can be porous and the pore size of the coating can be in the range of 0.01 μm to 1000 μm. The volume of porosity of the coating can be in the range of 5 vol % to 70 vol %. The thickness of the porous bioceramic coating can be in the range of 0.1 to 1000 μm. The pores of the bio-polymer/bioceramic matrix composite coating can be open and interconnecting.

The porous bioceramic matrix (a) can be selected from the group consisting of: hydroxyapatite, amorphous calcium phosphate (ACP), calcium metaphosphate, tricalcium phosphates, dicalcium phosphate dihydrate, calcium hydrogen phosphate, tetracalcium phosphates, heptacalcium decaphosphate, calcium pyrophosphate dihydrate, crystalline hydroxy apatite, poorly crystalline apatitic calcium phosphate, calcium pyrophosphate, monetite and octacalcium phosphate.

The invention is also directed to a bio-polymer/bioceramic matrix composite coating for a medical device comprising: (a) a porous bioceramic matrix; (b) at least one biocompatible polymer; and (c) at least one bioactive agent.

The bioceramic matrix (a) can be continuous phase. The biocompatible polymer can be continuous or discontinuous phase.

The porous bioceramic matrix coating can be made by a process selected from the group consisting of sol-gel coating, thermal spray coating, electro-chemical deposition, electrophoretic deposition, chemical vapor deposition, physical vapor deposition and biomimetic deposition. The pore size of the coating can be in the range of 0.01 μm to 1000 μm. The volume of porosity of the coating can be in the range of 5 vol % to 70 vol %. The thickness of the porous bioceramic coating is in the range of 0.1 μ to 1000 μm. The pores can be open and interconnecting.

The coating can be deposited on a medical device and the coating can cover at least a portion of the medical device.

The polymer can be impregnated into or infiltrated into the pores of the porous bioceramic matrix coating. The biocompatible polymer can be a biodegradable polymer or a non-biodegradable polymer.

The porous bioceramic matrix coating can be impregnated at least once with a polymer solution. The porous bioceramic matrix coating can multi-step impregnated with a polymer solution. The porous bioceramic matrix coating can be multi-step impregnated with dissimilar polymer solutions.

The bioactive agent can be selected from the group consisting of: anti-inflammatory agents, anti-cancer agents, antibiotics, anti-restenosis drugs, anti-thrombosis agents, antineoplastic agents and therapeutic combinations thereof. The bioactive agent of the bio-polymer/bioceramic matrix composite coating can be paclitaxel.

The medical device can be a stent. The medical device can be an implantable device or a surgical tool.

The pores in the coating can be created by including a burn-out additive in the coating and burning out the additive or by a gas-forming additive.

The biocompatible polymer can be non-biodegradable and can be selected from the group consisting of: polyether block amides (PEBA), polyoctenamers, polyolefins, ethylenic copolymers, ethylene vinyl acetate copolymers (EVA) and copolymers of ethylene with acrylic acid or methacrylic acid; thermoplastic polyurethanes (TPU) and polyurethane copolymers; metallocene catalyzed polyethylene (mPE), mPE copolymers, ionomers, and mixtures and copolymers thereof; and vinyl aromatic polymers and copolymers.

The biocompatible polymer can be biodegradable and can be selected from the group consisting of: biodegradable polylactic acid, polyglycolic acid, poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL), polyethylene oxide (PEO), polydioxanone (PDS), polypropylene fumarate, poly(ethyl glutamate-co-glutamic acid), poly(tert-butyloxy-carbonylmethyl glutamate), poly(carbonate-ester)s, polycaprolactone (PCL), polycaprolactone co-butylacrylate, polyhydroxybutyrate (PHBT) and copolymers of polyhydroxybutyrate, poly(phosphazene), poly(phosphate ester), poly(amino acid) and poly(hydroxy butyrate), polydepsipeptides, maleic anhydride copolymers, polyphosphazenes, polyiminocarbonates, cyanoacrylate, polyethylene oxide, hydroxypropylmethylcellulose, hyaluronic acid, chitosan and regenerate cellulose, and proteins such as gelatin and collagen, and mixtures and copolymers thereof.

The invention is also directed to a method of encapsulating a bioactive agent in a bio-polymer/bioceramic matrix composite coating comprising: (a) a porous bioceramic matrix coating; (b) at least one biocompatible polymer; and (c) at least one bioactive agent; said method being selected from the group consisting of: (i) immersing the composite bio-polymer/bioceramic matrix coating in a solution containing the bioactive agent; (ii) impregnating a solution of the biocompatible polymer and the bioactive agent into the porous bioceramic matrix coating; and (iii) multi-impregnating the composite coating by employing a combination of method (i) and method (ii).

The matrix composite coating after encapsulating the bioactive agent in the matrix composite coating can be coated with a thin polymer film. The film coated composite can be applied on a medical device.

The invention is also directed to a method of preparing a bio-polymer/bioceramic matrix composite comprising: (a) a porous bioceramic matrix of continuous phase; and (b) at least one biocompatible polymer of continuous or discontinuous phase, wherein the bioceramic matrix is made by a process selected from the group consisting of sol-gel coating, thermal spray coating, electro-chemical deposition, electrophoretic deposition, biomimetic deposition and shape and sintering.

The composite can incorporate a bioactive agent and can be deposited as a coating on a medical device and the coating can cover at least a portion of the medical device.

The polymer can be impregnated into the pores of the porous bioceramic matrix or it can be infiltrated into the pores of the porous bioceramic matrix.

The porous bioceramic matrix can be impregnated at least once with a polymer solution or it can be multi-step impregnated with a polymer solution. The porous bioceramic matrix coating can be multi-step impregnated with dissimilar polymer solutions.

### DRAWINGS

In drawings which illustrate specific embodiments of the invention, but which should not be construed as restricting the spirit or scope of the invention in any way:

FIGS. 1-10 illustrate salient features of the invention, and the effects of the application of the inventive PCMC coatings on surface of cardiovascular stents, in contrast to the behaviour of ceramic coatings only.

FIG. 1 provides the schematic comparison of the microstructure of polymer matrix composite and ceramic matrix composite (PCMC). The bioceramic fillers (such as fiber, particles, spheres) is a discontinuous phase and biopolymer is the continuous matrix of the composite in FIG. 1B. The biopolymer fillers (such as fiber, particles, spheres) is a continuous or discontinuous phase and bioceramic is the continuous matrix phase of the PCMC composite in FIG. 1D. The microstructural differences of two composites have significant impact on the biological and mechanical properties of materials.

FIG. 2 provides the schematic four (A, B, C, D) mechanisms of drug encapsulation in polymer-bioceramic matrix PCMC composite. Drug encapsulated in open pores of bioceramic matrix only (FIG. 2A) will be released through diffusion through the open pores, with release rate R1. Drug encapsulated in closed pores of bioceramic matrix only (FIG. 2A) will be released through resorption of the ceramic, with release rate R2. Drug encapsulated in the biopolymer residing in the open pores of bioceramic matrix, FIG. 2B, will be released through resorption of the polymer and diffusion through the open pores of the bioceramic, with release rate R3. Drug encapsulated in open pores and closed pores of bioceramic matrix, and in the biopolymer residing in the open pores of bioceramic matrix, FIG. 2C, will be released through resorption of the ceramic and the polymer, and diffusion through the open pores of the bioceramic, with release rate R4. The release rate R4 may be decreased to R5 by imposing a surface diffusion barrier of slowly- or non-resorbing polymer, FIG. 2D.

Although the drug release rates will vary with time, the rates generally can be ranked as follows: R5<R2<R3<R4<R1. Generally the drugs residing in biopolymer matrix are expected to release faster than these residing in the ceramic matrix. Therefore, the drug release profiles from PCMC can be engineered according to the specific clinical requirements, for short, medium and long term.

FIG. 3 provides the schematic comparison of biological and mechanical properties of bioceramics, biopolymer, and PCMC composites, in a “radar” diagram. The bioceramic matrix PCMC composites combine in the balanced fashion the best features of bioceramics and biopolymers, resulting in excellent biological and mechanical properties of PCMC in biomedical coating applications. These properties are relatively easy to adjust and optimize for the varying clinical requirements.

FIG. 4 illustrates the morphologies of (A) HAP porous coatings prepared using alcohol-based sol-gel solution with porogen agent (combustible polymer) to induce large fraction of porosity in HAP upon heat treatment and (B) HAP matrix composite coatings PCMC made by impregnating the polymer solution into HAP porous coating presented in FIG. 4A.

FIG. 5 illustrates the morphologies of (A) HAP porous coatings made by water-based sol-gel solution with porogen agent (combustible polymer) to induce large fraction of porosity in HAP upon heat treatment and (B) HAP matrix composite coatings made by impregnating the polymer solution into HAP porous coating shown in FIG. 5A.

FIG. 6 illustrates the morphologies of cross section of (A) HAP porous coatings showing with brittle fracture and (B) HAP-based composite PCMC coatings with ductile fracture as illustrated by arrow in FIG. 6B.

FIG. 7 illustrates the surface morphologies of bioceramic composite PCMC coatings produced of ECD-HAP coating impregnated with different concentration of PLGA solutions. It is shown that PLGA filled in most of the pores of ECD-HAP coating for 2 wt % PLGA solution and PLGA filled in the all pores of ECD-HAP coating for 4 wt % PLGA solution, however, the features of ECD coating surface can still be observed. The 6 wt % solution of PLGA filled in the all pores of ECD-HAP coating and additionally covered the surface of ECD coating such that the surface features of the ECD-HAP coating essentially disappeared.

FIGS. 8 and 9 illustrate the performance of PCMC coatings during expansion of coronary stents made of 316 stainless steel, and then coated with the respective PCMC. These two illustrations (FIG. 8, 9) are included, as expansion of coronary stent represents one of the most severe tests for coatings, as the stent undergoes strain of up to 10% in some regions. It is well known in the art that ceramics fail at strains on the order of 0.1%. The behaviour of PCMC during expansion of coronary stents illustrates the resilience of the coatings. That means, even if the ceramic backbone of the PCMC coating undergoes fracture, the fracture is contained by the polymeric component, to preserve the overall integrity of the coating.

FIG. 8 illustrates the expansion test of biopolymer-bioceramic composite PCMC coated stent, based on ECD-HAP impregnated with 2 wt % solution of PLGA. The ceramic component of the coatings was produced to have about 45 vol % of open porosity using ECD-HAP process. For the severe over-expansion shown in both tests, there is no PCMC cracking or separation for these stents.

FIG. 9 illustrates the expansion test of biopolymer-bioceramic composite PCMC coated stent, based on ECD-HAP impregnated with 4 wt % solution of PLGA. The ceramic component of the coatings was produced to have about 45 vol % of open porosity using ECD-HAP process. For severe over-expansion shown in both tests, there is no PCMC cracking or separation for these stents.

FIG. 10 illustrates expansion test of bioceramics only coated stent. The coatings were produced to have about 45 vol % of open porosity using ECD-HAP in the same process used for deposition of the ceramic component of the composite PCMC coatings on stents illustrated in FIGS. 8, 9. Even for typical expansion strain shown in this test, there is severe cracking and coating separation from the stent surface.

### DETAILED DESCRIPTION OF THE INVENTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

In the classical ceramic matrix composites, the primary goal of the polymer reinforcement is to provide toughness and to overcome the intrinsic brittleness and lack of reliability of the ceramics. The novel PCMC's according to the invention composites combine the desirable bioceramics with biopolymers to tailor properties such as strength, toughness and elasticity to meet structural system requirements, in addition to the inherent functional properties of the bio-polymer and bio-ceramic, such as biological properties and drug/protein delivery properties.

Many technologies are available to produce porous ceramics, wherein the porosity is open (i.e. accessible) porosity. For example, a porous ceramic matrix can be made by sol-gel processing with a surfactant, by mixing ceramics powders with porogens such as polymer particles or fibers as template and then sintering the product at high temperature.

The porous ceramic matrix can be subsequently infiltrated or impregnated by biopolymer solutions at room temperature. The open pores and voids of the ceramics matrix are filled with polymer solutions and then dried to form a ceramic matrix composite. Multi-infiltration processing may be required for increasing polymer content. The pore size and/or voids will be in range in 0.1 μm to 1000 μm and the polymer content will be 1-80 volume %.

For drug eluting applications, the drugs are incorporated into the pores of the bioceramic matrix and/or polymer solutions. Therefore, the pores and voids will serve as a drug carrying vehicle, and the drugs are encapsulated inside a bioceramic matrix. The drugs will release from the composite by diffusion and/or degradation of the biopolymer phase. The drugs releasing profile will be controlled by the porous structure and pore size of the matrix, polymer degradation rate, and interaction of bioceramics with the drugs.

For ceramics coating applications, the porous coatings can be fabricated by sol-gel processing with surfactants, i.e. Seed to Lu et al, “Continuous formation of supported cubic and hexagonal mesoporous films by sol-gel dip-coating”, in Nature (London) (1997), 389(6649), 364-368, Electro-Chemical Deposition (ECD) [i.e. Cheng et al, “Electrochemically assisted co-precipitation of protein with calcium phosphate coatings on titanium alloy”, in Biomaterials 25 (2004) 5395-5403], Electro-Phoretic Deposition (EPD) [i.e. Sridhar et al, “Preparation and characterization of electrophoretically deposited hydroxyapatite coatings on type 316L stainless steel”, in Corrosion Science (2003) 45(2), 237-252], and biomimetic coatings deposition [i.e. Costantini et al, “Hydroxyapatite coating of titanium by biomimetic method”, in Journal of Materials Science: Materials in Medicine (2002), 13(9), 891-894]. These publications are incorporated herein by reference. In order to increase the flexibility and reliability of the coatings, the porous coatings were impregnated with biopolymer solution to form biopolymer/ceramic matrix composites, at room or near-room temperatures. As the drug delivery vehicle, the drugs can be loaded and encapsulated inside the pores of ceramics matrix by impregnating with drug solution and polymer solution, individually, to control drug release profiles.

Beneficial drugs, proteins and therapeutic agents for the practice of the present invention include anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, proteins, DNA, and agents that interfere with endogenous vasoactive mechanisms.

The novel polymer-ceramic matrix composite (PCMC) with multi-functional properties can be used for a number of biomedical applications, such as, but not limited to, implantable devices, drug eluting stents, scaffolds, and tissue engineering.

The present invention is directed to a polymer-ceramic matrix composite material that comprises (a) a continuous bioceramic matrix (b) a biocompatible polymer and (c) a therapeutic bioactive agent.

A key inventive feature of the subject invention is that although the reinforcing polymer phase may be either continuous or discontinuous phase, the reinforced ceramic phase must be a continuous phase. The ceramic phase must be a continuous phase in order to provide a structural support in terms of stiffness and strength to the polymer filler phase. The primary goal of the reinforcement polymer phase is to provide toughness and to overcome the intrinsic brittleness and lack of reliability of the continuous ceramic phase. The PCMC composite is processed at room or near-room (37° C.) temperatures through impregnating a diluted solution of a polymer phase into the open pores of the ceramic phase. Both the polymer phase and ceramic phase of PCMC are bio-compatible. The novel PCMC composites therefore combine desirable bioceramics with biopolymers to tailor properties such as strength and elasticity, while maintaining desirable biological properties of the system, such as bio-degradability in biological environments. The new PCMC composite coatings can be used for biological and structural applications, as well as a vehicle for controlled release of biologically-active species such as drugs and proteins.

The bioceramic matrix composite (PCMC) provides structural support to the biopolymer filler phase the role of which is to provide toughness and to overcome the intrinsic brittleness and lack of reliability of the ceramic phase. The key feature of the invention is that the ceramic phase is a continuous phase. Therefore disintegration of the polymer phase, which takes place rapidly for bio-degradable polymers and less rapidly for other organic polymers, does not affect the integrity of the whole composite. Therefore, a variety of bio-polymer phases may be selected as fillers of the ceramic phase without substantially affecting the structural performance of the composite. This has significant impact on selection of the polymers for controlled drug delivery, e.g. rapidly dissolving polymers for rapid delivery of drugs may be selected without affecting composite integrity.

The PCMC material may be applied to deposit films, and coatings functioning in biological environments, e.g. films and coatings for implants. The bioceramic matrix composites can be used to improve the biocompatibility of metal implants and for better drug deliver vehicles, and can also function as fully resorbable scaffolds which provide temporary structures which are replaced as the body rebuilds tissue.

Materials for medical implants devices must be non-toxic. As many materials will chemically interact when exposed to tissue or body fluids, the products of such chemical interaction must also be non-toxic. This in many cases is difficult to avoid entirely because it is well known that metallic implants will release harmful metal ions to body fluids. Irrespective of this disadvantage, metals are used because of their irreplaceable structural properties such as strength and stiffness.

Although almost every living organism requires certain structural support from the tissue to maintain the functionality of the organism (the larger the organism, the more critical the structural support becomes), nature never “selected” metals to provide that necessary support. This is because the chemical functionality of metals, in particular the toxicity towards living tissue, overrides their potential benefits as structural materials. Instead, by selection, organo-ceramic composites largely constitute what is known as “hard tissue”, e.g. bone or tooth. This is the combination of calcium phosphate ceramic with the natural tissue such as collagen, provides an excellent structural material, which is at the same time an entirely bio-compatible material, bio-resorbable without any adverse effects towards the host tissue.

The present invention follows this basic lesson from nature and inventively proposes the use of a porous, continuous ceramic matrix of calcium phosphate, in particular HAP, as a carrier of bio-polymer, both components being entirely bio-compatible and bio-resorbable without any adverse effects towards the host tissue. Thus the whole composite is entirely bio-compatible and bio-resorbable without any adverse effects towards the host tissue.

Many polymers will degrade and the products of degradation will be toxic or trigger allergic/inflammatory reaction of the tissue. However, researchers are frequently forced to select such polymers for implants because of a number of reasons, such as structural properties. The present invention enables the selection of polymers which do not produce adverse reaction of tissue upon resorption, even if the polymer phase lacks the required structural properties such as strength. This is because the bio-ceramic phase, such as calcium phosphate (in particular HAP) provides sufficient structural support, whereas the organic polymer phase may be selected entirely for its biological/chemical advantages. It is well known that calcium phosphate (in particular HAP) is entirely bio-compatible and provides no adverse reaction from living tissue upon resorption.

According to another aspect of the present invention, a therapeutic-agent-releasing medium is provided, which comprises: (a) an implantable or insertable medical device; (b) a release or drug or biological agent over at least a portion of the implantable or insertable medical device; and (c) a therapeutic agent. Upon implantation or insertion of the device into a patient, the rate of release of the therapeutic agent is controlled by microstructure of the bioceramic matrix, the ceramic matrix degradation rate, the biopolymer degradation rate, and the diffusion rate of the degradation products and the biologically active agents (drugs, proteins) through the porosity channels of the ceramic matrix. This is in contrast to the drug delivery systems based on (i) bio-polymer only, or (ii) biopolymer combined with ceramic wherein the ceramic is a discontinuous phase. In both cases (i) and (ii) the drug release is controlled by degradation of the polymer, without the additional factors of controlling the release of the therapeutic agent by bioceramic matrix, the ceramic matrix degradation rate, and the diffusion rate of the degradation products and the biologically active agents (drugs, proteins) through the porosity channels of the ceramic matrix.

In a preferred embodiment of the invention, the biodegradation rate of biopolymer phase will be faster than that of a bioceramic matrix. Consequently, the products of degradation, and biologically active agents, will be released from the PCMC body only after diffusion through the network of open porosity in the bio-ceramic matrix. This diffusion process allows for long-time delivery of steady level dosage of the agents. Additionally, in the initial period after implantation of the composite bio-material, the polymer phase increases the composite toughness and reliability, and only at a later stage contributes to the release of the biological agents from pores or voids of porous ceramic matrix through the biodegradation and diffusion of bioactive agents. The porous ceramic matrix provides a biocompatible surface and structure for drug delivery.

An important aspect of the subject invention is the possibility of using such composite materials as coatings for implants. The novel PCMC bioceramic matrix composite coatings overcome the disadvantages of brittleness of entirely ceramic materials and increase flexibility and reliability, which is especially useful for flexible substrates, such as stents. During stent implanation, the deformation and stresses due to stent expansion may cause serious damage to the bioceramic coatings on the stent because of their brittleness. Thus the fully ceramic coating may suffer defects such as cracks, delamination, and debris release. Also, biologically active agents such as drugs or proteins, are difficult to retain or otherwise encapsulate within a fully ceramic matrix. The bioceramics matrix composite increases the flexibility and bonding strength of coatings, allowing for controlled encapsulation and release of the biological agents. Suitable ceramic matrix calcium phosphates include, but are not limited to, hydroxyapatite, amorphous calcium phosphate, calcium metaphosphate, tricalcium phosphates, dicalcium phosphate dihydrate, calcium hydrogen phosphate, tetracalcium phosphates, heptacalcium decaphosphate, calcium pyrophosphate dihydrate, crystalline hydroxy apatite, poorly crystalline apatitic calcium phosphate, calcium pyrophosphate, monetite and octacalcium phosphate. All these phosphates may have partially crystalline, or amorphous calcium phosphate structures. The degree of crystallinity allows additional control of the resorption rate of the composite, i.e. less crystalline ceramics will resorb faster.

Ceramics in a number of forms and compositions are currently in use or under consideration for use as biomaterials. Titinia, mullite, silica, alumina and zirconia are among the bioinert ceramics used for prosthetic devices. Porous ceramics such as calcium phosphate-based materials are used for filling bone defects. The ability to control porosity and solubility of some ceramic materials offers the possibility of their use as drug delivery systems.

In the subject invention, the PCMC is directed to overcoming the main drawback of monolithic and films ceramics, namely their brittleness. The PCMC's are referred to as inverse composites, which is to say that the failure strain of the matrix is lower than the failure strain of the ceramics, whereas it is the reverse in most polymer matrix composites. In order to prevent an early failure of the brittle ceramics when the matrix starts to microcrack, ceramic matrix bonding will be controlled during processing. PCMC's according to the invention are tough materials and display a high failure stress when the bonding between polymers and ceramic matrix is not too strong or too weak.

In the subject invention, porous bioceramic coatings can be made by burning-out additives, which can be represented by any combustible material that is economically justifiable. Bioceramic slurry or sol-gel mixed with burning-out additives are coated on substrates by dipping, spinning, and spraying. The porosity of coatings with burning-out additives depends on their type, content, and the grain size. A maximum content of such additives is limited by the fact of loosening and abrupt decrease in strength of material. Such ceramics should be fired in an oxidizing medium until complete burning-out of the additive. The method of introducing burn-out additives makes it possible to produce bioceramic coatings with porosity up to 20 vol %-65 vol %.

In this invention, several methods of chemical formation of pores in suspensions to achieve porous coatings, are used. For example, gas-forming additives can be used to ensure formation of a large volume of gases, and a uniform release of gas within a prescribed temperature interval. The additives should not be toxic. Among numerous potential chemical reactions involving gas formation, the ones practically used are reactions between carbonates and acids. The process of formation of a cellular mixture in chemical formation of pores depends on many factors: the suspension viscosity, the temperature, type, content, and dispersion of the solid gas-forming agent, the type of acid and its content, and the presence and content of a stabilizer for the swelled mixture. This method provides for production of ceramics with high and super high porosity based on various initial materials, which is used in thermal insulation and heat-shielding. However, the porosity formed this way is frequently a closed porosity, which does not allow impregnation of secondary phases such as bio-polymer phase into the pores.

According to one aspect of this invention, porous bioceramic matrix coatings are deposited on the surface of implantable medical devices by sol-gel processing with polymer surfactant porogen, electro-chemical deposition, electrophoresis deposition, biomimetic deposition, composite sol-gel processing, spray coating, spin coating, dip coating, or plasma spray coatings. In this invention, all commercial available porous ceramic coatings can be used as composite ceramic matrixes.

The porous matrices of bioceramics may be macroporous or microporous. Microporous matrices typically have pores in the range from about 0.1 μm to about 100 microns in size, while macroporous matrices typically have pores in the range from about 100 to about 1000 microns in size. In certain embodiments the pore size in a given range is substantially uniform. The pores in the matrix account for the void volume. Such void volume may be from about 10% to about 90%. The pores are typically interconnecting, and in some cases to a substantial degree. The pores may form an open-cell configuration in some embodiments. In embodiments where the void volume constitutes a substantial portion of the matrix volume, the pores are typically close together.

In this invention, the mechanisms for drug encapsulation and controlled release include, but are not limited to, the following:

(1) The bioactive agents are encapsulated into the bioceramic matrix composite coatings PCMC through dipping a porous bioceramic matrix coating into a drug solution, then removing the excess of drug solution by spinning. Subsequently, biopolymers are impregnated into the micropores of the bioceramic coating in the composite PCMC structure. The drug is immobilized inside the mesopores of the porous bioceramic coatings, and may be released by diffusion through the bioceramic matrix and biopolymer barrier, degradation of biopolymer and bioceramic matrix. The drug eluting rate is slow and persists in long term. In this variant of the invention, the drug material is not combined with the polymer material.

(2) The bioactive agents are encapsulated by impregnating a biopolymer and drug mixture solution into a bioceramic matrix to form bioceramic matrix composite PCMC. Extra solution is removed by spinning. The drug is released by diffusion and degradation of biopolymer. By comparison with conventional drug and biopolymer coatings, there is no biopolymer debris released during the biopolymer degradation, of a size that is larger than the pores of the ceramic matrix. This is because the biopolymer and drug are immobilized inside microporous and mesoporous structures. Also, the drug eluting rate is much slower that that of normal biopolymer coatings.

(3) The bioactive agents are encapsulated through multi-drug encapsulation, with different release rates and functions by a combination of the two processing methods 1, 2 above. For example, paclitaxel can be encapsulated into a porous bioceramic matrix by dipping porous coatings into a paclitaxel-alcohol solution. Such drug-loaded paclitaxel porous bioceramic coatings are then impregnated by a mixture solution of biopolymer and rapamycin to form a bioceramic matrix composite. The release rate of paclitaxel inside the ceramic matrix is much slower that that of a drug in biopolymer phase.

(4) In order to meet the special requirements of long term drug release (e.g. up to one year), the thin polymer film as the drug diffusion barrier can be deposited on the surface of the bioceramic matrix composite coatings. The drugs in both of the ceramic matrix and biopolymer phase must go though the barrier and slow the release rate.

The above example methods for use of PCMC for drug delivery illustrate the multiple possibilities of the PCMC system, which are impossible to achieve through use of polymer only, or ceramic only, or a composite in which the ceramic is a dispersed phase within the polymer phase.

The polymeric materials used for making PCMC composite coatings may comprise any biocompatible polymer suitable for use in implantable or insertable medical devices. The biocompatible polymer may be substantially non-biodegradable or biodegradable. The term “biocompatible” describes a material that is not substantially toxic to the human body, and that does not significantly induce inflammation or other adverse response in body tissues. Biocompatible polymers include essentially any polymer that is approved or capable of being approved by Food and Drug Administration (FDA) for use in humans or animals when incorporated in or on an implantable or insertable medical device.

Non-biodegradable polymers include, but are not limited to, polyether block amides (PEBA), polyoctenamers, polyolefins, ethylenic copolymers, ethylene vinyl acetate copolymers (EVA) and copolymers of ethylene with acrylic acid or methacrylic acid; thermoplastic polyurethanes (TPU) and polyurethane copolymers; metallocene catalyzed polyethylene (mPE), mPE copolymers, ionomers, and mixtures and copolymers thereof; and vinyl aromatic polymers and copolymers.

Biodegradable polymers include, but are not limited to, polylactic acid, polyglycolic acid, poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL), polyethylene oxide (PEO), polydioxanone (PDS), polypropylene fumarate, poly(ethyl glutamate-co-glutamic acid), poly(tert-butyloxy-carbonylmethyl glutamate), poly(carbonate-ester)s, polycaprolactone (PCL), polycaprolactone co-butylacrylate, polyhydroxybutyrate (PHBT) and copolymers of polyhydroxybutyrate, poly(phosphazene), poly(phosphate ester), poly(amino acid) and poly(hydroxy butyrate), polydepsipeptides, maleic anhydride copolymers, polyphosphazenes, polyiminocarbonates, cyanoacrylate, polyethylene oxide, hydroxypropylmethylcellulose, hyaluronic acid, chitosan and regenerate cellulose, and proteins such as gelatin and collagen, and mixtures and copolymers thereof, among others.

The therapeutic agent for use in composite coatings of the present invention can be any pharmaceutically acceptable therapeutic agent which is approved or capable of being approved by Food and Drug Administration (FDA) for use in humans or animals when incorporated in or on an implantable or insertable medical device. As noted above, preferred therapeutic agents include anti-inflammatory agents, anti-cancer agents, antibiotics; anti-restenosis drugs anti-thrombosis agents, antineoplastic agents and combinations thereof.

The bioactive agents include, but are not limited to, phenylbutazone, gentomycin, vancomycin, indomethacin, naproxen, ibuprofen, flubiprofen, diclofenac, dexmethasone, prednisone and prednisolone, gentomycin, vancomycin, rapamicin, paclitaxel, actinomycin, sirolimus, everolimus, tacrolimus, dexamethasone, mycophenolic acid, and heparin.

In this invention, the amount of therapeutic agent present in bioceramic matrix will depend upon the efficacy of the therapeutic agent employed, the length of time during which the medical device is to remain implanted, as well as the rate at which the bioceramic matrix or barrier layer releases the therapeutic agent in the environment of the implanted medical device. Thus, a device that is intended to remain implanted for a longer period will generally require a higher percentage of the therapeutic agent. Similarly, a bioceramic matrix that provides faster rate of release of the therapeutic agent may require a higher percentage of the therapeutic agent. One skilled in the art can readily determine an appropriate therapeutic agent content to achieve the desired outcome.

### EXAMPLES

General Example of PCMC Processing

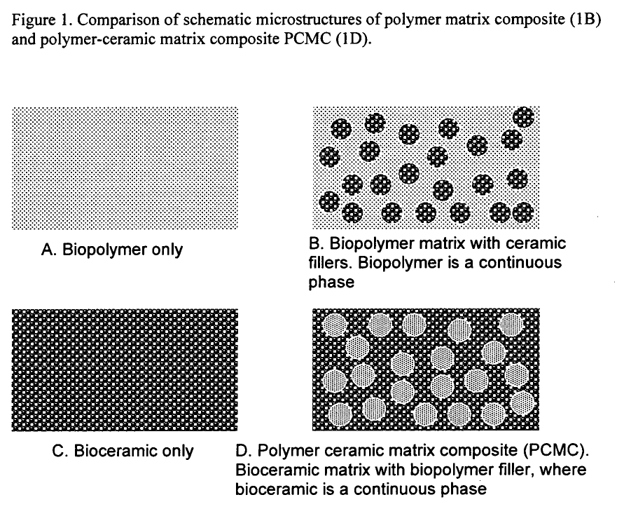

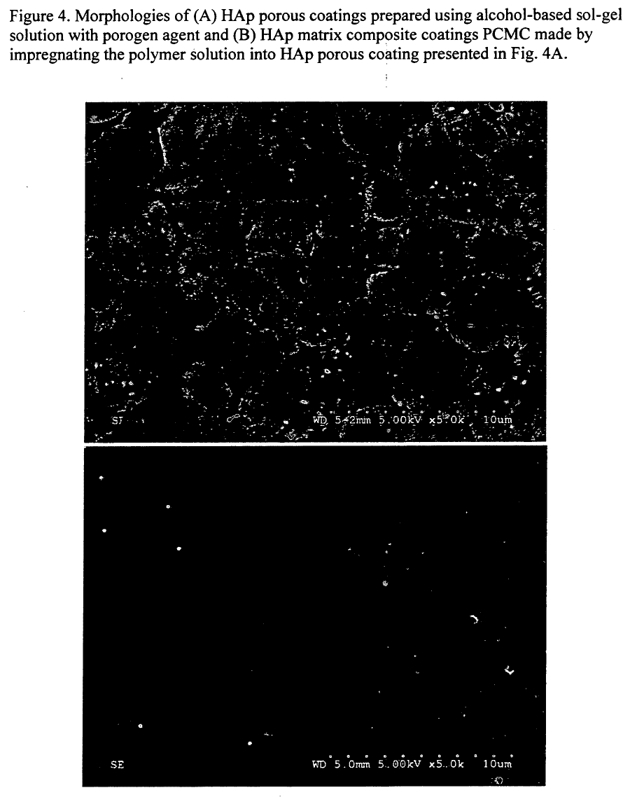

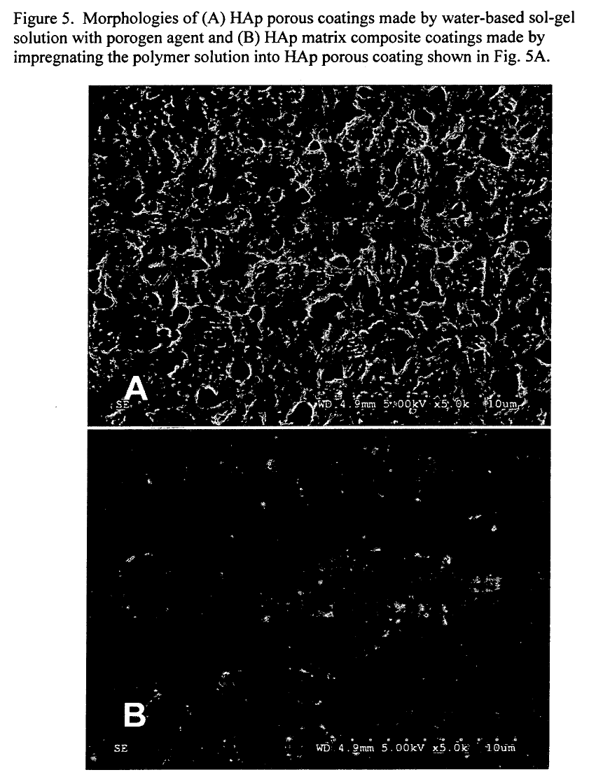

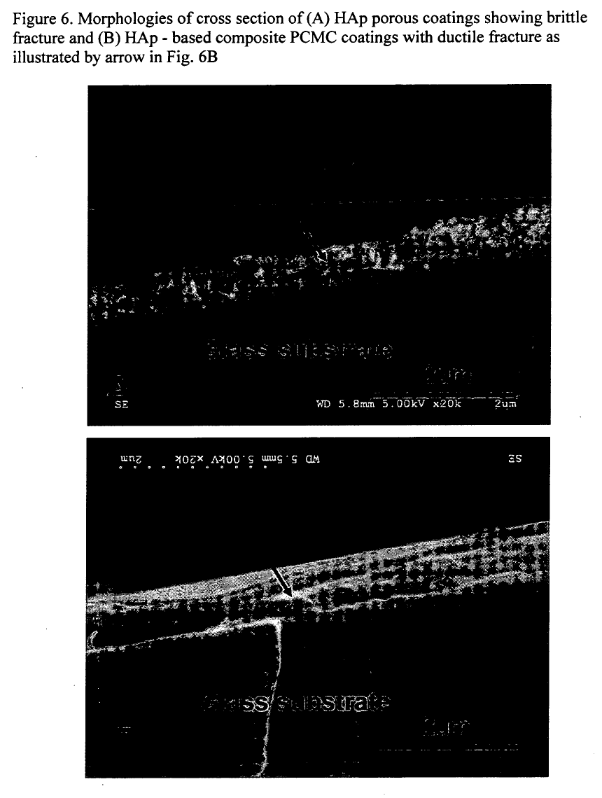

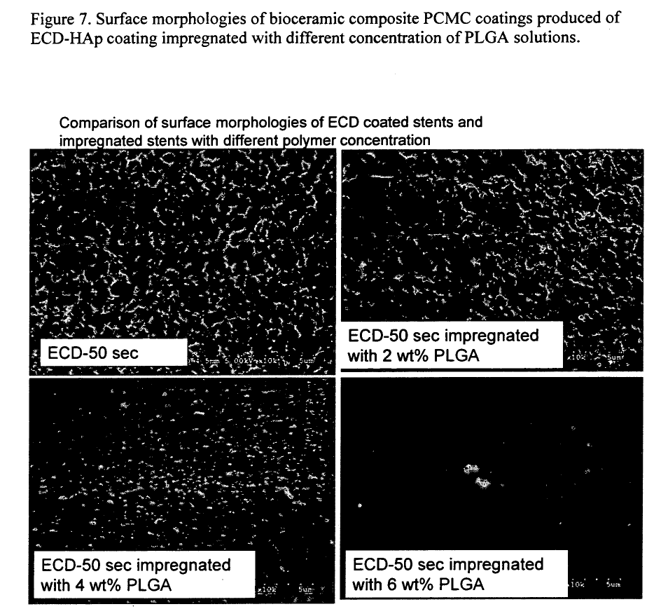

This example presents the general processing steps, the processing variants, and the resulting properties of PCMC. The coatings microstructures and performance is illustrated through FIGS. 1-10. The specific details of the specific processes to achieve the specific desired properties of PCMC coatings are provided in Examples 1-10 below.

In the general PCMC process, a porous ceramic coating is deposited on a substrate. There are may well known techniques for depositing porous ceramic coatings, and some of these techniques are presented in more details in the examples 1-10 below. The coating open porosity (i.e. porosity accessible to outside gases or liquids) is generally in the range of 1-80 vol %, more desirably in the range 10-50 vol %. The coating thickness is in the range of about 0.1-1000 μm, more desirably in the range 0.5-10 μm. The coating phase is bio-ceramic or bio-glass, more desirably calcium phosphate such as HAP. The distinctive feature of the current invention is that the ceramic phase is a continuous phase, as illustrated in FIG. 1C. As such, the ceramic phase acts as a back-bone of the coating system and thus during dissolution of the organic polymer phase the structural integrity of the coating is retained.

In general Process 1, the bio-polymer is dissolved in a suitable Solvent A such as water or alcohol. The polymer concentration in the solvent depends on the type of the polymer/solvent system. The general requirement is that the system viscosity and wettability of the ceramic at room or near-room temperature is low enough to allow polymer penetration of the pores in the ceramic down to 0.1 μm range, preferably down to 0.05 μm range. The concentration of the polymer in the solvent can be in the range of 0.1-50 wt %, preferably in the range 1-10 wt %.

The bio-polymer solution is impregnated into the porous ceramic phase through simple immersion, or through vacuum-assisted or pressure-assisted impregnation. Sufficient time is allowed for the solution to penetrate the 0.1 μm pores in the ceramic, the time and the required pressure depending on the solution viscosity, wettability of the ceramic, and pore size distribution in the ceramic. Afterwards, the sample is removed from the solution, excess solution is removed, e.g. through spinning, and the solvent is removed by evaporation at room or near-room temperature. The resulting PCMC resembles the microstructure illustrated in FIG. 1D. In a variant of this general Process 1, a molten thermoplastic polymer may be used instead of the polymer solution. This variant however precludes use of any temperature-sensitive additives in the process, such as drugs. Processes 2 and 3 below are preferred for such temperature-sensitive additives.

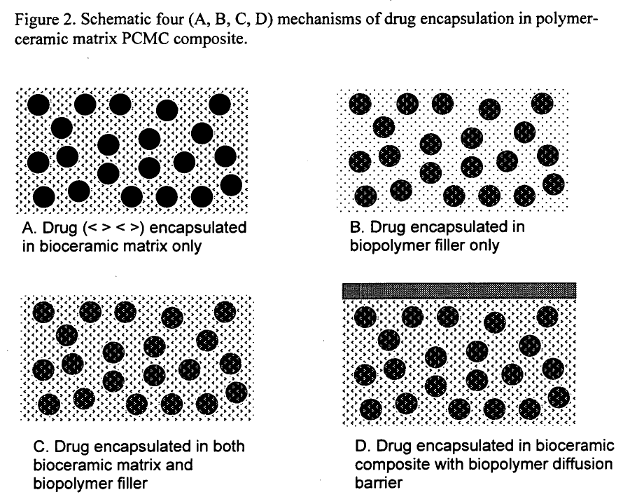

In a variant of the above process herein named Process 2, a drug D1 or protein P1 is dissolved in Solvent A together with the polymer, and impregnated into the porous ceramic matrix, followed by excess solution removal and solvent removal as described above. The resulting microstructure is typically like the one illustrated in FIG. 2B.

In a variant of the above process herein named Process 3, a drug D2 or protein P2 is dissolved in Solvent B, and impregnated into the porous ceramic matrix, followed by excess solution removal and solvent removal as described above. Subsequently, the polymer is dissolved in Solvent A, and this solution is impregnated into the remaining porosity within the ceramic. If the drug D1 or protein P1 is not soluble in Solvent A, there is no carry-over of D1 or P1 into the polymer phase during the impregnation process, and the resulting microstructure is like the one illustrated in FIG. 2A.

Processes 2 and 3 may be combined to result in encapsulation of various drugs and proteins both in the polymer phase and the ceramic phase, as illustrated in FIG. 2C. The excess polymer film may be left on the PCMC to further enhance mechanical properties of the composite, and add further control to the delivery of drug from the PCMC vehicle, as illustrated in FIG. 2D.

Specific Illustrative Examples of PCMC Processing

### Example 1

### Poly(lactic acid)—Hydroxyapatite (HAP) Matrix PCMC Composite Coatings by Sol-Gel Processing

The porous HAP coatings were fabricated through a sol-gel route. There are a number of sol-gel routes to HAP, as disclosed in the scientific and patent literature. In this particular example, the inventors have followed the route disclosed previously by one of the co-authors (TT) in U.S. Pat. No. 6,426,114, issued Jul. 30, 2002, the contents of which are incorporated herein by reference. In this route, as quoted from U.S. Pat. No. 6,426,114, “phosphite sol was hydrolysed in a water-ethanol mixture (a concentration of 3M) in a sealed beaker until the phosphite was completely hydrolysed (which is easily recognized by loss of a characteristic phosphite odour), at ambient environment. A Ca salt (2M) was then dissolved in anhydrous ethanol, and the solution was then rapidly added into the hydrolysed phosphite sol. The sol was left at ambient environment for 8 hours, followed by drying in an oven at 60° C. As a result of this process, a white gel was obtained. For the sol containing Ca/P ratio required to produce HA, the gel showed a pure (single phase) apatitic structure with a Ca/P ratio of 1.666, identical to stoichiometric HA, after calcining at a temperature as low as 350° C. Varying the Ca/P ratio allows other calcium phosphates, such as dicalcium phosphate (Ca/P=1) or tricalcium phosphate (Ca/P=1.5), to be obtained. A coating produced using this process, and applied to Ti substrate, showed sufficient adhesive strength after curing at a temperature <450° C. The coating was crack-free and porous.”

There are many other known sol-gel routes to porous HAP. The sol-gel coatings may also be deposited on substrates through numerous routes, such as dip-coating, spin-coating, spray-coating, aerosol-coating, and others. For the purpose of the current example, spray coating processing was selected. The coatings were dried at 100° C. for 20 min, and fired at 500° C. for 30 min. The firing process decomposes all the precursors used in sol preparation, aids in formation of HAP structure (either crystalline or amorphous, depending on temperature of heat treatment), partially removes porosity in the structure, and as well, removes other organic additives which may be used, such as polymer surfactants.

The thickness of the resulting porous sol-gel HAP coatings is typically in range of 0.2-2 μm with porosity in range 10-30 vol %, majority of which (>90%) is open porosity, e.g. accessible to impregnation. The pore size is typically in range of 0.01 to 0.1 μm. The coating processed in this particular example had thickness of 0.4 μm and porosity of about 25 vol %.

The porous sol-gel HAP coating was impregnated by bio-polymer through the following route. 1 g of poly (lactic acid) was dissolved into 10 g methylcholine. The porous HAP coatings on stents were impregnated with polymer solution for 4 hours, in which time the solution will have reached all the pores of the coating and interface of substrate and coatings. The extra solution was removed by centrifuge (spin) processing, followed by drying at 37° C. for 60 minutes. This process resulted in deposition of the polymer within the pores of the ceramic matrix. As diluted solution of polymer was used, the pores were only partially filled with the polymer. In this particular example, about 20% of the available volume within the pores was filled. In order to increase the polymer content, multi-step impregnation is necessary.

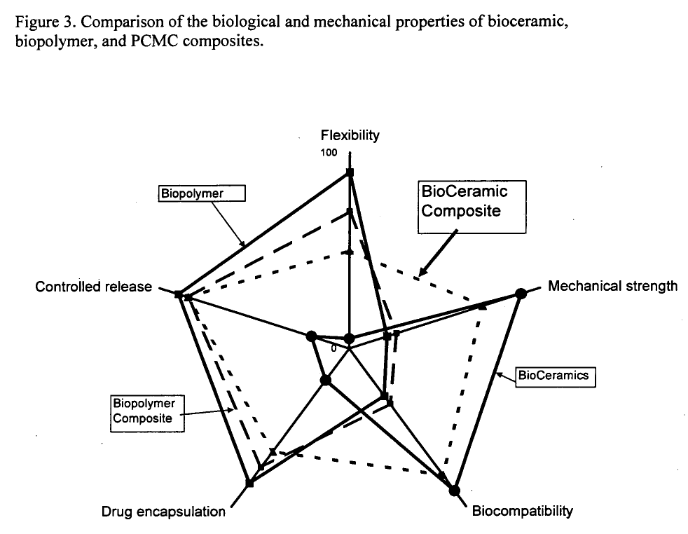

The resulting Poly(lactic acid) Hydroxyapatite (HAP) Matrix PCMC composite coatings have advantageous properties resulting from combination of the properties of the biopolymer and the properties of the continuous network of porous bioceramics, as illustrated in FIG. 3. These include (i) mechanical properties, such as mechanical flexibility (i.e. enhanced strain to failure), strong interfacial bonding, high fracture toughness; and (ii) biological properties, such as high biocompatibility and no toxic products of biodegradation.

The resulting Poly(lactic acid) Hydroxyapatite (HAP) Matrix PCMC composite coatings are suitable for coating implants such as hip implant, dental implants, stents, and many other implants. The particular combination of biocompatibility and strain tolerance makes the PCMC composites particularly suitable for implants undergoing strain and deformation during implantation, such as stents.

### Example 2

### Poly(lactic acid)-Drug-Hydroxyapatite (HAP) Matrix PCMC Composite Coatings by Sol-Gel Processing

The porous HAP coatings were fabricated and deposited on implant surface through sol-gel route, as described in Example 1. The porous sol-gel HAP coating was impregnated by bio-polymer-drug mix through the following route. 1 g of poly (lactic acid) and 0.2 g Rapamycin were co-dissolved into 10 g methylcholine. The porous HAP coatings were impregnated with polymer and drug solution for 4 hours, in which time the solution will have reached all the pores of the coating and interface of substrate and coatings. The extra solution was removed by centrifuge (spin) processing, followed by drying at 37° C. for 60 minutes. This process resulted in deposition of the drug and polymer within the pores of the ceramic matrix. About 20-50 μg of drug can be deposited within the pores of such processed PCMC, per 1 cm² of the coating. In this particular example, 34 μg of drug was deposited within the PCMC per 1 cm² of the coating.

The resulting Poly(lactic acid)-drug-Hydroxyapatite (HAP) Matrix PCMC composite coatings have advantageous properties resulting from combination of the properties of the biopolymer, the drug and the properties of the continuous network of porous bioceramics, as illustrated in FIG. 3. These include (i) mechanical properties, such as mechanical flexibility (i.e. enhanced strain to failure), strong interfacial bonding, high fracture toughness; (ii) biological properties, such as high biocompatibility and no toxic products of bio-degradation; and (iii) drug delivery properties, such as long term drug eluting profile controlled by degradation rate of the polymer AND transport through porosity network in the ceramic.

The resulting Poly(lactic acid)-Drug-Hydroxyapatite (HAP) Matrix PCMC composite coatings are suitable for coating implants such as hip implant, dental implants, stents, and many other implants. The particular combination of biocompatibility, drug delivery and strain tolerance makes the PCMC composites particularly suitable for implants undergoing strain and deformation during implantation, such as stents.

However, as the sol-gel coatings thickness typically does not exceed about 1 μm, similarly the PCMC coatings thickness also typically does not exceed about 1 μm (unless additional polymer membrane is deposited as illustrated in FIG. 2D), and therefore the overall volume of the pores available to carry drugs or proteins is relatively small. Alternative processing routes to achieve thicker PCMC coatings suitable for carrying larger amounts of drugs are described in Examples 3-7.

### Example 3

### Poly(lactic acid)-Drug-Hydroxyapatite (HAP) Matrix PCMC Composite Coatings by Plasma Spray Processing

Deposition of porous HAP coatings by plasma spraying is well known and documented in literature. We have used one of the standard processing routes to deposit 110 μm thick, 30 vol % porous (including 8 vol % closed porosity and 22 vol % open porosity) HAP coating. The ceramic HAP matrix was a continuous matrix used for impregnation to produce PCMC. The coating was impregnated with drug-biopolymer as described in Example 2. The resulting 110 μm thick PCMC was suitable for implants of relatively simple surface features or pattern, such as hip implants or dental implants, and unsuitable for complex deforming implants such as stents. The coatings were advantageous over the pure ceramic HAP coatings typically used for hip or dental implants because of advantageous (i) biological properties, such as high biocompatibility and no toxic products of bio-degradation; and (ii) drug delivery properties, such as and long term drug eluting profile controlled by degradation rate of the polymer AND transport through porosity network in the ceramic. About 200-1000 μg of drug can be deposited within the pores of such processed PCMC, per 1 cm² of the coating. In this particular example we have deposited 330 μg of drug within Poly(lactic acid)-drug-Hydroxyapatite (HAP) Matrix PCMC composite coating.

### Example 4

### Poly(lactic acid)-Drug-Hydroxyapatite (HAP) Matrix PCMC Composite Coatings by Electro-Chemical Deposition

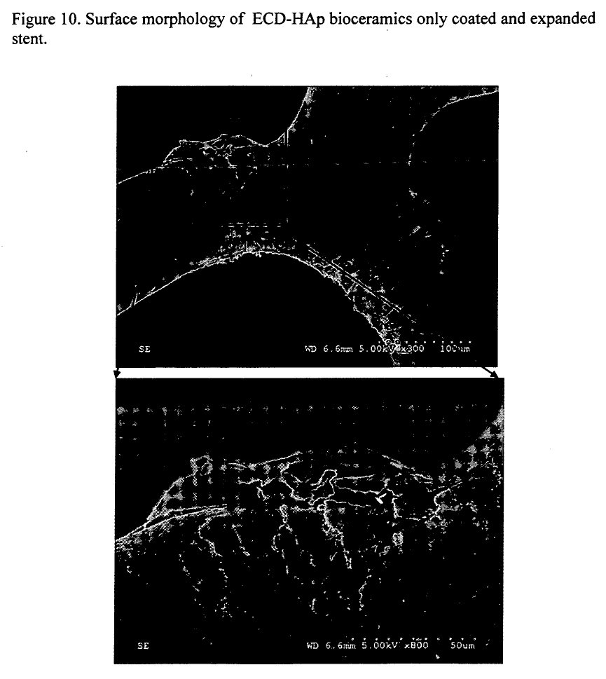

Porous HAP coatings were fabricated through Electro-Chemical Deposition (ECD). The electrolyte solution used for the electrochemically assisted precipitation of calcium phosphate consisted of 0.042 mol Ca(NO₃)₂ and 0.025 mol NH₄H₂PO₄ prepared using distilled water. The pH of the solution was approximately 4.2, and the solution temperature was maintained at 65° C. The precipitation was carried out galvanostatically at a cathodic current of 0.6 mA/cm² for 0.5-10 min. Following precipitation, the specimen was rinsed with distilled water and air dried for use. Thickness of the coatings was in range of 0.2-10 μm, typically 0.5-3 μm, and porosity in range 30-70 vol %. The distribution of pore size was typically in range of 0.1 to 10 μm. In this particular example, the coating was 0.7 μm thick, with 45 vol % of pores in the range of 0.05-0.3 μm. Although good absorbents of drugs and polymers, the porous ECD-HAP coatings have relatively poor mechanical performance. This is illustrated in FIG. 10 wherein the ECD-HAP coating only was deposited on stent surface, and then the stent expanded. Significal mechanical damage to the coating, including separation of the coating from the stent surface, results.

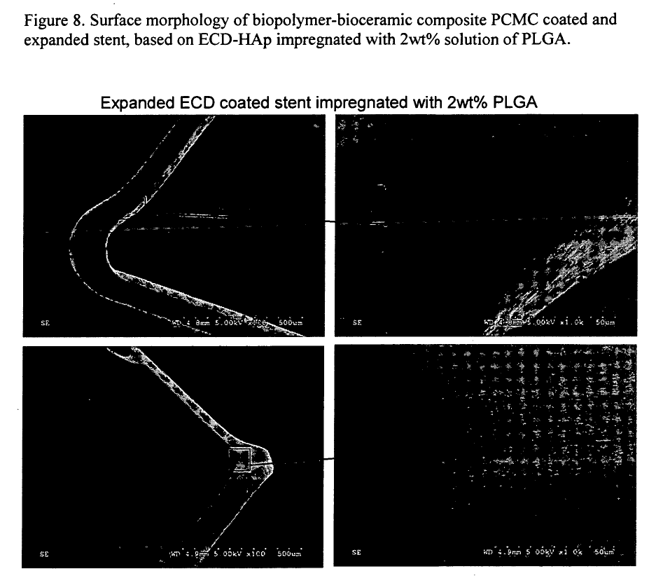

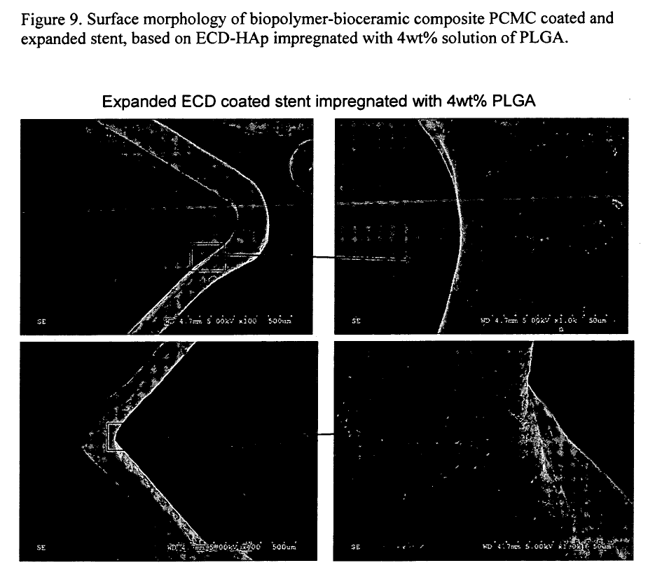

The porous ECD-HAP coating was impregnated by bio-polymer-drug mix through the route of Example 2. As the coating is thicker and more porous as compared to the sol-gel coating (presented in Examples 1, 2), about 100-300 μg of drug can be deposited within the pores of such processed PCMC, per 1 cm² of the coating. In this particular example, we have deposited 60 μg of drug per 1 cm² of the PCMC coating. As the ECD route to HAP coating provides good control of the coating uniformity and thickness on complex substrates, the technology is suitable for stents (as opposed to the plasma spray route in Example 3). FIGS. 8 and 9 illustrate the expansion test of such biopolymer-bioceramic composite PCMC coated stent, based on ECD-HAP impregnated with 2 wt % (FIG. 8) and 4 wt % (FIG. 9) solution of PLGA. Dramatic difference of the PCMC coatings behaviour, as compared to ECD-HAP coating only, is evident upon comparison of FIGS. 8, 9 and 10. For the severe over-expansion shown in both tests shown in FIGS. 8 and 9, there is no PCMC cracking or separation for these stents.

### EXAMPLE 5

### Poly(lactic acid)-Drug-Hydroxyapatite (HAP) Matrix PCMC Composite Coatings by Electro-Phoretic Deposition

Porous HAP coatings were fabricated through Electro-Phoretic Deposition EPD. The suspensions of nano-HAP particles were prepared adding 5 g of HAP powders to 400 ml of ethanol. The suspensions were dispersed ultrasonically during 30 min with an ultrasonic vibrator. The suspension was rested during 24 h to eliminate, by sedimentation, the bigger particles. Voltage of 10 V was applied for depositing the coatings at 10 second. The EPD coatings was sintered at 550° C. for 20 min. As the EPD route to HAP coating provides good control of the coating uniformity and thickness on complex substrates, the technology is suitable for stents (as opposed to the plasma spray route in Example 3). Thickness of the coatings was in range of 0.5-5 μm, typically 1.0-3 μm, and porosity in range 20-50 vol %. The distribution of pore size was typically in range of 0.1-2 μm. In this particular example the coating was 1.2 μm thick, with 35 vol % of pores in the range of 0.1-0.3 μm. The porous EPD-HAP coating was impregnated by bio-polymer-drug mix through the route of Example 2. As the coating is thicker and more porous as compared to the sol-gel coating (presented in Examples 1 and 2), about 100-200 μg of drug can be deposited within the pores of such processed PCMC, per 1 cm² of the coating. In this particular example, we have deposited 55 μg of drug per 1 cm² of the PCMC coating.

### Example 6

### Natural Polymer-Hydroxyapatite (HAP) Matrix PCMC Composite Coatings by Electro-Chemical Deposition

Porous HAP coatings were fabricated through Electro-Chemical Deposition ECD, as in Example 4. 1 g of chitosan, a bio-polymer derived from natural sources (chitin) was dissolved into 10 g of water. The porous sol-gel HAP coating was impregnated by bio-polymer through the route of Example 1. The resulting Chitosan (Collagen)-Hydroxyapatite (HAP) Matrix PCMC composite coatings have advantageous properties resulting from combination of the properties of the natural polymer and the properties of the continuous network of porous bioceramics. These include (i) mechanical properties, such as mechanical flexibility (i.e. enhanced strain to failure), strong interfacial bonding, high fracture toughness; and (ii) biological properties, such as high biocompatibility and no toxic products of bio-degradation. In a variant of this process, collagen was used instead of chitosan, leading to similar properties of PCMC coating.

The resulting PCMC composite coatings are suitable for coating implants such as hip implant, dental implants, stents, and many other implants. The particular combination of biocompatibility and strain tolerance makes the PCMC composites particularly suitable for implants undergoing strain and deformation during implantation, such as stents.

### Example 7

### Drug Encapsulated in Bioceramics Matrix Only as Illustrated in FIG. 2A

Porous HAP coating was deposited on stent as described in the above Example 1. The HAP porous coating stents was impregnated with 2 wt % paclitaxel-methanol solution for 20 min and then extra solution was removed by high speed spinning, and then the solvent dried in oven for 2 hours. The paclitaxel filled mostly the mesopores (<0.1 μm) and partially the larger micropores (>0.1 μm) of the coating. Subsequently the paclitaxel loaded stents were impregnated by 10 wt % PLGA acetone solution, primarily into the larger (still accessible micropores) and then the extra solution removed by spinning. The biopolymer PLGA inside the pores of the ceramic coating is free of drug and results in a PCMC composite with flexibility and strain tolerance, similarly as illustrated in FIGS. 8 and 9. The polymer filler provided additionally the diffusion barrier for controlling drug release profiles.

### Example 8

### Drug Encapsulated in Biopolymer Filler Only as Illustrated in FIG. 2B

Porous HAP coating was deposited on a stent as in the above Example 2. 1 g PLGA was dissolved into 10 g methylcholine together with 0.1 g paclitaxel. The porous HAP coatings on stents were impregnated with polymer and drug solution for 2 hours, in which time the solution will have reached and filled all the pores in the ceramic coating, and also the interface between the substrate and the coatings. The extra solution was then removed by spinning and then the solvent dried in an oven for 2 hours. The PLGA/HAP Matrix composite coatings on stents have advance properties of combination of biopolymer and bioceramics, such as mechanical flexibility of coatings, strong interfacial bonding, high biocompatibility, and long term drug eluting characteristics.

### Example 9

### Drug Encapsulated in Both Bioceramics Matrix and Biopolymer Filler—FIG. 2C

Porous HAP coating was deposited on stent as in the above Example 2. The HAP porous coating stents was impregnated into 2 wt % paclitaxel methanol solution for 20 min and then removed extra solution by high speed spinning, then dried in an oven for 2 hours. The paclitaxel was filled into mesopores and partial into large pore micropores. The paclitaxel loaded stents were subsequently impregnated with 10 wt % PLGA acetone solution containing 2 wt % Rapamycin and then the extra solution removed by spinning. The biopolymer PLGA filled inside pores provides extra flexibility for HAP coatings and diffusion barrier for controlling drug release profiles. Rapamycin in the polymer phase will release much faster than that of paclitaxel only in the HAP phase.

### Example 10

### Drug Encapsulated in Bioceramic Composite with Biopolymer Diffusion Barrier as Illustrated in FIG. 2D.

As illustrated in the above Example 9, different drugs were encapsulated into the polymer and HAP phases. In order to add further controls for drug release profile, e.g. to further slow down the drug release rate, a functional diffusion barrier was deposited on the surface of the composite PCMC coating. In this particular example, a 2 μm thick PLGA (85:15) layer was deposited on the PCMC surface by spin-coating. Rapamycin release from such modified PCMC coating was sustained for 3-5 months and paclitaxel for 6-12 months.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A bio-polymer/bioceramic matrix composite coating comprising: (a) a porous bioceramic matrix of continuous phase; and (b) at least one biocompatible polymer of continuous or discontinuous phase.

2. A bio-polymer/bioceramic matrix composite coating as claimed in claim 1 wherein a bioactive agent is incorporated in the composite coating.

3. A bio-polymer/bioceramic matrix composite coating as claimed in claim 1, wherein said porous bioceramic matrix is made by a process selected from the group consisting of sol-gel coating, thermal spray coating, electro-chemical deposition, electrophoretic deposition, biomimetic deposition and shape and sintering.

4. A bio-polymer/bioceramic matrix composite coating as claimed in claim 1, wherein the coating is porous and the pore size of the coating is in the range of 0.01 μm to 1000 μm.

5. A bio-polymer/bioceramic matrix composite coating as claimed in claim 4, wherein volume of porosity of the coating is in the range of 5 vol % to 70 vol %.

6. A bio-polymer/bioceramic matrix composite coating as claimed in claim 4, wherein the thickness of the porous bioceramic coating is in the range of 0.1 to 1000 μm.

7. A bio-polymer/bioceramic matrix composite coating as claimed in claim 4, wherein the pores are open and interconnecting.

8. A bio-polymer/bioceramic matrix composite coating as claimed in claim 1, wherein the porous bioceramic matrix (a) is selected from the group consisting of:
hydroxyapatite, calcium metaphosphate, tricalcium phosphates, dicalcium phosphate dihydrate, calcium hydrogen phosphate, tetracalcium phosphates, heptacalcium decaphosphate, calcium pyrophosphate dihydrate, crystalline hydroxy apatite, poorly crystalline apatitic calcium phosphate, calcium pyrophosphate, monetite and octacalcium phosphate.

9. A bio-polymer/bioceramic matrix composite coating as claimed in claim 1, wherein the bioceramic matrix is hydroxyapatite.

10. A bio-polymer/bioceramic matrix composite coating for a medical device comprising: (a) a porous bioceramic matrix;(b) at least one biocompatible polymer; and (c) at least one bioactive agent.

11. A bio-polymer/bioceramic matrix composite coating as claimed in claim 10 wherein the bioceramic matrix (a) is continuous phase.

12. A bio-polymer/bioceramic matrix composite coating as claimed in claim 10 wherein the biocompatible polymer is continuous or discontinuous phase.

13. A bio-polymer/bioceramic matrix composite coating as claimed in claim 10, wherein said porous bioceramic matrix coating is made by a process selected from the group consisting of sol-gel coating, thermal spray coating, electrochemical deposition, electrophoretic deposition, chemical vapor deposition, physical vapor deposition and biomimetic deposition.

14. A bio-polymer/bioceramic matrix composite coating as claimed in claim 10, wherein the pore size of the coating is in the range of 0.01 μm to 1000 μm.

15. A bio-polymer/bioceramic matrix composite coating as claimed in claim 10, wherein volume of porosity of the coating is in the range of 5 vol % to 70 vol %.

16. A bio-polymer/bioceramic matrix composite coating as claimed in claim 14, wherein the thickness of the porous bioceramic coating is in the range of 0.1 μm to 1000 μm.

17. A bio-polymer/bioceramic matrix composite coating as claimed in claim 14, wherein the pores are open and interconnecting.

18. A bio-polymer/bioceramic matrix composite coating as claimed in claim 10, wherein the porous bioceramic matrix (a) is selected from the group consisting of:
hydroxyapatite, amorphous calcium phosphate, calcium metaphosphate, tricalcium phosphates, dicalcium phosphate dihydrate, calcium hydrogen phosphate, tetracalcium phosphates, heptacalcium decaphosphate, calcium pyrophosphate dihydrate, crystalline hydroxy apatite, poorly crystalline apatitic calcium phosphate, calcium pyrophosphate, monetite and octacalcium phosphate.

19. A bio-polymer/bioceramic matrix composite coating as claimed in claim 10 wherein the bioceramic matrix coating is hydroxyapatite.

20. A bio-polymer/bioceramic matrix composite coating as claimed in claim 10, wherein the coating is deposited on a medical device and the coating covers at least a portion of the medical device.

21. A bio-polymer/bioceramic matrix composite coating as claimed in claim 10, wherein the polymer is impregnated into the pores of the porous bioceramic matrix coating.

22. A bio-polymer/bioceramic matrix composite coating as claimed in claim 10, wherein the polymer is infiltrated into the pores of the porous bioceramic matrix coating.

23. A bio-polymer/bioceramic matrix composite coating as claimed in claim 10, wherein the biocompatible polymer is a biodegradable polymer.

24. A bio-polymer/bioceramic matrix composite coating as claimed in claim 10, wherein the biocompatible polymer is a non-biodegradable polymer.

25. A bio-polymer/bioceramic matrix composite coating as claimed in claim 10, wherein the porous bioceramic matrix coating is impregnated at least once with a polymer solution.

26. A bio-polymer/bioceramic matrix composite coating as claimed in claim 10, wherein the porous bioceramic matrix coating is multi-step impregnated with a polymer solution.

27. A bio-polymer/bioceramic matrix composite coating as claimed in claim 10, wherein the porous bioceramic matrix coating is multi-step impregnated with dissimilar polymer solutions.

28. A bio-polymer/bioceramic matrix composite coating as claimed in claim 10, wherein the bioactive agent is selected from the group consisting of:
anti-inflammatory agents, anti-cancer agents, antibiotics, anti-restenosis drugs, anti-thrombosis agents, antineoplastic agents and therapeutic combinations thereof.

29. A bio-polymer/bioceramic matrix composite coating as claimed in claim 10, wherein the bioactive agent is paclitaxel.

30. A bio-polymer/bioceramic matrix composite coating as claimed in claim 20, wherein the medical device is a stent.

31. A bio-polymer/bioceramic matrix composite coating as claimed in claim 20, wherein the medical device is an implantable device or a surgical tool.

32. A bio-polymer/bioceramic matrix coating as claimed in claim 14 wherein the pores in the coating are created by including a burn-out additive in the coating and burning out the additive.

33. A bio-polymer/bioceramic matrix coating as claimed in claim 14 wherein the pores in the matrix are created by a gas-forming additive.

34. A bio-polymer/bioceramic matrix coating as claimed in claim 10 wherein the biocompatible polymer is non-biodegradable and is selected from the group consisting of:
polyether block amides (PEBA), polyoctenamers, polyolefins, ethylenic copolymers, ethylene vinyl acetate copolymers (EVA) and copolymers of ethylene with acrylic acid or methacrylic acid; thermoplastic polyurethanes (TPU) and polyurethane copolymers; metallocene catalyzed polyethylene (mPE), mPE copolymers, ionomers, and mixtures and copolymers thereof; and vinyl aromatic polymers and copolymers.

35. A bio-polymer/bioceramic matrix coating as claimed in claim 10 wherein the biocompatible polymer is biodegradable and is selected from the group consisting of:
biodegradable polylactic acid, polyglycolic acid, poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL), polyethylene oxide (PEO), polydioxanone (PDS), polypropylene fumarate, poly(ethyl glutamate-co-glutamic acid), poly(tert-butyloxy-carbonylmethyl glutamate), poly(carbonate-ester)s, polycaprolactone (PCL), polycaprolactone co-butylacrylate, polyhydroxybutyrate (PHBT) and copolymers of polyhydroxybutyrate, poly(phosphazene), poly(phosphate ester), poly(amino acid) and poly(hydroxy butyrate), polydepsipeptides, maleic anhydride copolymers, polyphosphazenes, polyiminocarbonates, cyanoacrylate, polyethylene oxide, hydroxypropylmethylcellulose, hyaluronic acid, chitosan and regenerate cellulose, and proteins such as gelatin and collagen, and mixtures and copolymers thereof.

36. A method of encapsulating a bioactive agent in a bio-polymer/bioceramic matrix composite coating comprising:
(a) a porous bioceramic matrix coating;
(b) at least one biocompatible polymer; and
(c) at least one bioactive agent; said method being selected from the group consisting of:
(i) immersing the composite coating bio-polymer/bioceramic matrix in a solution containing the bioactive agent;
(ii) impregnating a solution of the biocompatible polymer and the bioactive agent into the porous bioceramic matrix coating; and
(iii) multi-impregnating the composite coating by employing a combination of method (i) and method (ii).

37. A method as claimed in claim 36 wherein the matrix composite coating after encapsulating the bioactive agent in the matrix composite coating is coated with a thin polymer film.

38. A method as claimed in claim 36 wherein the composite coating is deposited on a medical device.

39. A method of preparing a bio-polymer/bioceramic matrix composite comprising: (a) a porous bioceramic matrix of continuous phase; and (b) at least one biocompatible polymer of continuous or discontinuous phase, wherein the bioceramic matrix is made by a process selected from the group consisting of sol-gel coating, thermal spray coating, electro-chemical deposition, electrophoretic deposition, biomimetic deposition and shape and sintering.

40. A method as claimed in claim 39 wherein the composite incorporates a bioactive agent and the composite is deposited as a coating on a medical device and the coating covers at least a portion of the medical device.

41. A method as claimed in claim 39 wherein the polymer is impregnated into the pores of the porous bioceramic matrix.

42. A method as claimed in claim 39 wherein the polymer is infiltrated into the pores of the porous bioceramic matrix.

43. A method as claimed in claim 39 wherein the porous bioceramic matrix is impregnated at least once with a polymer solution.

44. A method as claimed n claim 39 wherein the porous bioceramic matrix is multi-step impregnated with a polymer solution.

45. A method as claimed in claim 39 wherein the porous bioceramic matrix coating is multi-step impregnated with dissimilar polymer solutions.
